(19) United States
(12) Patent Application Publication
(10) Pub. No.: US 2026/0306441 A1
(43) Pub. Date: Oct. 8, 2026

(54) MEDICAMENT CONTAINING ENTEROPEPTIDASE INHIBITOR AS ACTIVE INGREDIENT, AND METHOD FOR PREVENTING AND OR TREATING POLYCYSTIC KIDNEY DISEASE CONTAINING ADMINISTERING THE SAME

(71) Applicants: SCOHIA PHARMA, INC., Fujisawa-shi (JP); NATIONAL UNIVERSITY CORPORATION HOKKAIDO UNIVERSITY, Sapporo-shi (JP)

(72) Inventors: Jun SUGAMA, Fujisawa (JP); Yusuke MORITOH, Fujisawa (JP); Masanori WATANABE, Fujisawa (JP); Fumihiko HATTANDA, Sapporo-shi (JP); Takuro KAWAMURA, Sapporo-shi (JP); Saori NISHIO, Sapporo-shi (JP)

(73) Assignees: SCOHIA PHARMA, INC., Fujisawa-shi (JP); NATIONAL UNIVERSITY CORPORATION HOKKAIDO UNIVERSITY, Sapporo-shi (JP)

(21) Appl. No.: 19/637,127

(22) Filed: Apr. 2, 2026

(30) Foreign Application Priority Data
Apr. 3, 2025 (JP) 2025-061712

(51) Int. Cl. A61K 31/343; A61P 13/12

(52) U.S. Cl. CPC A61K 31/343; A61P 13/12

(57) ABSTRACT

A medicament containing an enteropeptidase inhibitor as an active ingredient, for preventing and/or treating polycystic kidney disease; a method for preventing and/or treating polycystic kidney disease, containing administering an effective amount of an enteropeptidase inhibitor to a subject requiring the administration are provided as some embodiments of the present invention. They are highly effective in suppressing and/or improving cyst formation, or suppressing and/or improving kidney volume increase.

### TECHNICAL FIELD

The present invention provides, as embodiments thereof, a medicament containing an enteropeptidase inhibitor as an active ingredient, for suppressing and/or improving cyst formation, or suppressing and/or improving kidney volume increase based on the suppression and/or improvement of cyst formation, in a subject suffering from polycystic kidney disease; a medicament containing an enteropeptidase inhibitor as an active ingredient, for preventing and/or treating polycystic kidney disease, or polycystic liver disease as a complication thereof; a method for suppressing and/or improving cyst formation, or suppressing and/or improving kidney volume increase based on the suppression and/or improvement of cyst formation, in a subject suffering from polycystic kidney disease, containing administering an effective amount of an enteropeptidase inhibitor to the subject; a method for preventing and/or treating polycystic kidney disease, containing administering an effective amount of an enteropeptidase inhibitor to a subject requiring the administration; and the like, and is useful in the pharmaceutical field.

### BACKGROUND ART

Polycystic kidney disease is a hereditary disease characterized by the development of numerous cysts in both kidneys, with the largest number of patients among the hereditary kidney diseases. It is broadly classified into autosomal dominant polycystic kidney disease (ADPKD) and autosomal recessive polycystic kidney disease (ARPKD) based on the causative gene, but most patients have ADPKD. Based on epidemiological surveys, the number of ADPKD patients in Japan is estimated as 31,000 (one in 4,033 people). ADPKD is primarily caused by PKD1 and PKD2 genes, with about 85% of patients developing the disease due to a mutation in the PKD1 gene. Polycystin 1/2, which is the gene product of PKD1/2, is a membrane protein localized in the cilia of renal tubular epithelial cells. It senses the flow of urine within the tubules and regulates tubular diameter by allowing Ca²⁺ to flow into the cells. In ADPKD, loss of polycystin 1/2 function leads to a decrease in Ca²⁺ concentration within renal tubular epithelial cells. As a result, abnormal cell proliferation, tubular dilation, and increased cystic fluid secretion occur, leading to the progressive formation of numerous cysts in both kidneys, and a gradual decrease in glomerular filtration rate (GFR), which is an indicator of renal function. Most ADPKD patients are asymptomatic until around age 30-40, but symptoms associated with renal cyst enlargement appear thereafter, and about half of patients reach end-stage renal failure by age 70. The most common subjective symptom is kidney pain (60%) caused by cystic bleeding, cystic infection, urinary tract stones, and extension of renal capsule. Other known complications include hypertension, polycystic liver disease, heart valve disease, cerebral aneurysms, and the like.

At present, there is no fundamental cure for ADPKD, and the only treatment drug for suppressing the progression is the vasopressin type 2 receptor antagonist tolvaptan, which is covered by insurance. However, the therapeutic effect thereof is not sufficient and, in Japan, prescription thereof is restricted to cases where the total bilateral kidney volume is 750 mL or more and the rate of kidney volume increase is 5% or more per year, due to concerns about serious side effects. A post-marketing surveillance study, which collected case data from 1672 clinical cases, showed an incidence of side effects in 46.47%, with hypernatremia reported in 5.56% and liver dysfunction or liver damage reported in 12.74%, of patients. Furthermore, 60 patients (3.59%) discontinued treatment due to insufficient efficacy, and 318 patients (19.0%) discontinued treatment due to adverse events. Furthermore, because this drug induces rapid water diuresis due to its mechanism of action, at least the initial administration should be performed under hospitalization, and appropriate fluid intake should be instructed to prevent dehydration. For hypertension as a complication, salt intake restriction and administration of angiotensin receptor blockers or angiotensin-converting enzyme inhibitors are recommended. On the other hand, there is no treatment for polycystic liver disease, and tolvaptan is ineffective.

As described above, the existing drug tolvaptan has efficacy and safety issues, and the number of eligible patients is limited. Therefore, the development of a new therapeutic agent with a novel mechanism of action that can be applied to a wide range of patients is necessary.

In cystic epithelial cells of ADPKD patients, the expression of mTOR (mammalian Target Of Rapamycin), which is a serine/threonine kinase involved in cell proliferation and metabolism, is elevated, and excessive activation thereof is known to be involved in the progression of ADPKD (Non Patent Literature 1). In fact, mTOR inhibitors suppress cyst formation and renal function decline in animal models of polycystic kidney disease (Non Patent Literature 2). Therefore, clinical trials using mTOR inhibitors have been conducted in ADPKD patients, but no efficacy was observed, and the development has been discontinued (Non Patent Literatures 3-4). In the everolimus trial, total kidney volume significantly decreased at the 1-year mark of treatment, but the significant difference disappeared at the 2-year mark of treatment, and no improvement in GFR, which is an indicator of renal function, was observed. However, mTOR inhibitors have a high incidence of side effects, and in this trial, 33% of the active drug group discontinued the trial. Furthermore, the occurrence of side effects affecting renal hemodynamics, such as edema, suggests that drug-induced side effects may have worsened renal function. Moreover, the fact that the dosage could not be increased sufficiently due to the concerns about side effects is considered to be one factor contributing to the lack of efficacy (Non Patent Literature 2). Therefore, a clear conclusion regarding whether mTOR inhibition is useful in the treatment of ADPKD has not been reached due to issues with the compound.

On the other hand, from the gene expression analysis of cystic tissue from ADPKD patients and renal tissue from healthy individuals, it has been reported that in ADPKD patients, mitochondrial dysfunction leads to compensatory glycolysis, resulting in increased mTOR activity due to decreased AMP-activated protein kinase (AMPK) activity (Non Patent Literature 5). Therefore, based on the concept of suppressing glucose utilization or utilizing ketone bodies, the usefulness of calorie-restricted diets and ketogenic diets as dietary therapy for ADPKD patients has been investigated in recent years, and a certain inhibitory effect on the increase in total kidney volume and the decrease in GFR has been found (Non Patent Literature 6). Both dietary therapies are assumed to involve the suppression of mTOR via AMPK activation as the mechanism, and these test results suggest that mTOR may be one of the promising therapeutic targets for ADPKD. However, continuous dietary restriction places a significant burden on patients and leads to a decline in quality of life. Therefore, implementation thereof in clinical practice is considered to be limited in ADPKD requiring long-term therapeutic intervention, and intervention through drug therapy, which places less burden on patients, is desired.

In recent years, a link between the regulation of mTOR activity by amino acids and ADPKD has been reported. Among the nutrients contained in food, branched-chain amino acids (BCAAs), particularly leucine, are known to activate mTOR. Furthermore, decreased activity of the BCAA metabolic pathway has been reported in ADPKD patients, suggesting that BCAAs tend to accumulate and mTOR activation by BCAAs is more likely to occur (Non Patent Literature 5). In fact, when BCAAs were dissolved in the drinking water of ADPKD model mice and BCAA intake was increased, the mTOR signaling in the kidneys was significantly activated and the formation of renal cysts was promoted. This suggests that amino acids absorbed from the diet may be involved in cyst formation (Non Patent Literature 7). Therefore, inhibiting the activation of mTOR signaling by dietary amino acids could be effective in treating ADPKD. However, the problem remains as to how to achieve a decrease in amino acid levels through drug intervention rather than dietary restriction and how to link this to treatment, and a solution has not yet been found.

Enteropeptidase is a transmembrane serine protease specifically expressed on the duodenal lumen side, and has the physiological action of cleaving and activating trypsinogen in pancreatic juice into trypsin. Activated trypsin degrades proteins in food into amino acids, which are then absorbed into the body. Therefore, enteropeptidase is the rate-limiting enzyme in the digestion and absorption of proteins. It is considered that the inhibition of the enzymatic activity of enteropeptidase suppresses the digestion and absorption of proteins in food. Therefore, enteropeptidase inhibitors may be effective in treating polycystic kidney disease by inhibiting amino acid absorption after meals and inhibiting the activation of mTOR signaling by amino acids.

However, while various enteropeptidase inhibitors have been reported to date (Patent Literatures 1-6) and usefulness thereof in anti-diabetic use, anti-obesity use, and the like has been described, there are no suggestions or reports indicating their effectiveness on polycystic kidney disease.

### CITATION LIST

### Patent Literature

### [Patent Literature 1]

WO 2015/122187

### [Patent Literature 2]

WO 2016/158788

### [Patent Literature 3]

WO 2016/104630

### [Patent Literature 4]

WO 2011/071048

### [Patent Literature 5]

WO 2019/088270

### [Patent Literature 6]

WO 2019/216742

### Non Patent Literature

### [Non Patent Literature 1]

Proc Natl Acad Sci USA. 2006, 103 (14): 5466-5471

### [Non Patent Literature 2]

Kidney Res Clin Pract. 2012, 31 (3): 132-138

### [Non Patent Literature 3]

N Engl J Med. 2010, 363 (9): 830-840

### [Non Patent Literature 4]

N Engl J Med. 2010, 363 (9): 820-829

### [Non Patent Literature 5]

Int J Mol Sci. 2024, 25 (13): 7173

### [Non Patent Literature 6]

Cell Rep Med. 2023, 4 (11): 101283

### [Non Patent Literature 7]

Kidney Int. 2017, 92 (2): 377-387

### SUMMARY OF INVENTION

### Technical Problem

As described above, a method for preventing or treating ADPKD with a medicament has not yet been established, and the development of a preventive or therapeutic agent with an action mechanism different from that of existing drugs and having high clinical efficacy has been demanded. In addition, an effective method for preventing or treating ARPKD has not been established and, in the actual medical situation, there has been a similar demand as in ADPKD.

Therefore, the present invention aims to provide a medicament for preventing and/or treating polycystic kidney disease, or polycystic liver disease as a complication thereof; a method for preventing and/or treating polycystic kidney disease, or polycystic liver disease as a complication thereof; and the like.

### Solution to Problem

Under these circumstances, the present inventors conducted intensive studies and, as described in detail in the Examples section below, found that in tests using an enteropeptidase inhibitor, the medicament suppressed mTOR activity in the kidney, suppresses hypertrophy and cyst formation of the kidney, and surprisingly also suppresses hypertrophy and cyst formation of the liver. Based on this new finding, the present inventors have completed the present invention, which uses an enteropeptidase inhibitor for the prevention or treatment of polycystic kidney disease, or for the prevention or treatment of polycystic liver disease as a complication thereof.

The present invention is specifically described in the following based on embodiments, but the present invention is not limited thereto. Those of ordinary skill in the art can change the embodiments of the present invention into various embodiments without departing from the significance of the present invention. Such changes are also encompassed in the present invention.
[1] A medicament comprising an enteropeptidase inhibitor as an active ingredient, for the suppression and/or improvement of cyst formation, or the suppression and/or improvement of kidney volume increase based on the suppression and/or improvement of cyst formation, in subjects suffering from polycystic kidney disease.
[2] The medicament of the above-mentioned [1], for the improvement of renal function.
[3] The medicament of the above-mentioned [1] or [2], for preventing and/or treating polycystic kidney disease.
[4] The medicament of any of the above-mentioned [1] to [3], for preventing and/or treating polycystic kidney disease, and polycystic liver disease as a complication thereof.
[5] The medicament of the above-mentioned [3] or [4], wherein the polycystic kidney disease is ADPKD (autosomal dominant polycystic kidney disease) or ARPKD (autosomal recessive polycystic kidney disease).
[6] The medicament of any of the above-mentioned [1] to [5], wherein the enteropeptidase inhibitor is a compound described in WO 2015/122187, WO 2016/158788, WO 2016/104630, WO 2011/071048, WO 2019/088270, or WO 2019/216742, or a salt thereof, or a hydrate thereof.
[7] The medicament of any of the above-mentioned [1] to [5], wherein the enteropeptidase inhibitor is a compound described in WO 2015/122187, or a salt thereof, or a hydrate thereof.
[8] The medicament of any of the above-mentioned [1] to [5], wherein the enteropeptidase inhibitor is a compound represented by the formula (I):

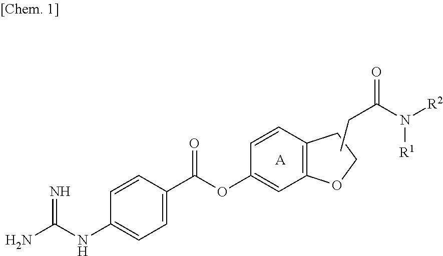

wherein
ring A is a benzene ring optionally substituted by 1 to 3 substituents selected from a halogen atom and a C₁₋₆ alkyl group;
R¹ is a hydrogen atom, or a C₁₋₆ alkyl group substituted by COOH, and
R² is a C₁₋₆ alkyl group substituted by 1 or 2 COOH, and further optionally substituted by SO₃H,
   described in WO 2015/122187, or a salt thereof, or a hydrate thereof.
[9] The medicament of any of the above-mentioned [1] to [5], wherein the enteropeptidase inhibitor is a compound selected from the group consisting of
N-(((3S)-6-((4-carbamimidamidobenzoyl)oxy)-2,3-dihydro-1-benzofuran-3-yl)acetyl)-L-aspartic acid (Example 1),
N-(((3R)-6-((4-carbamimidamidobenzoyl)oxy)-2,3-dihydro-1-benzofuran-3-yl)acetyl)-L-aspartic acid (Example 2), and
N-((6-((4-carbamimidamidobenzoyl)oxy)-4-methyl-2,3-dihydro-1-benzofuran-3-yl)acetyl)-L-aspartic acid (Example 3),
   described in WO 2015/122187, or a salt thereof, or a hydrate thereof.
   [10] The medicament of any of the above-mentioned [1] to [5], wherein the enteropeptidase inhibitor is N-(((3S)-6-((4-carbamimidamidobenzoyl)oxy)-2,3-dihydro-1-benzofuran-3-yl)acetyl)-L-aspartic acid or a salt thereof, or a hydrate thereof.
   [11] The medicament of any of the above-mentioned [1] to [5], wherein the enteropeptidase inhibitor is N-(((3S)-6-((4-carbamimidamidobenzoyl)oxy)-2,3-dihydro-1-benzofuran-3-yl)acetyl)-L-aspartic acid hydrate (WO 2015/122187, Example 12).
   [12] A method for suppressing and/or improving cyst formation, or suppressing and/or improving kidney volume increase based on the suppression and/or improvement of cyst formation, in a subject suffering from polycystic kidney disease, comprising administering an effective amount of an enteropeptidase inhibitor to the subject.
   [13] The method of the above-mentioned [12], for the improvement of kidney function.
   [14] A method for preventing and/or treating polycystic kidney disease, comprising administering an effective amount of an enteropeptidase inhibitor to a subject requiring the administration.
   [15] The method of the above-mentioned [14], for preventing and/or treating polycystic kidney disease and polycystic liver disease as a complication thereof.

### Advantageous Effects of Invention

The present invention provides, as embodiments thereof, a medicament containing an enteropeptidase inhibitor as an active ingredient, for suppressing and/or improving cyst formation, or suppressing and/or improving kidney volume increase based on the suppression and/or improvement of cyst formation, in a subject suffering from polycystic kidney disease; a medicament containing an enteropeptidase inhibitor as an active ingredient, for preventing and/or treating polycystic kidney disease, or polycystic liver disease as a complication thereof; a method for suppressing and/or improving cyst formation, or suppressing and/or improving kidney volume increase based on the suppression and/or improvement of cyst formation, in a subject suffering from polycystic kidney disease, containing administering an effective amount of an enteropeptidase inhibitor to the subject; a method for preventing and/or treating polycystic kidney disease, containing administering an effective amount of an enteropeptidase inhibitor to a subject requiring the administration; and the like.

### DESCRIPTION OF EMBODIMENTS

Some embodiments of the present invention include the following forms.
[A] A medicament comprising an enteropeptidase inhibitor as an active ingredient, for the suppression and/or improvement of cyst formation, or the suppression and/or improvement of kidney volume increase based on the suppression and/or improvement of cyst formation, in subjects suffering from polycystic kidney disease.
[A-1] The medicament of the above-mentioned [A], for the improvement of renal function.
   (The above is also collectively referred to as “Embodiment A”.)
[B] A medicament containing an enteropeptidase inhibitor as an active ingredient, for preventing and/or treating polycystic kidney disease.
[B-1] The medicament of the above-mentioned [B], for preventing and/or treating preventing and/or treating polycystic kidney disease, and polycystic liver disease as a complication thereof.
[B-2] The medicament of the above-mentioned [B] or [B-1], wherein the polycystic kidney disease is ADPKD (autosomal dominant polycystic kidney disease) or ARPKD (autosomal recessive polycystic kidney disease).
   (The above is also collectively referred to as “Embodiment B”.)

The above-mentioned embodiments are described below by specifically defining the technical meaning of the terms used to specify the technical matters of each embodiment, but the present invention is not limited to these embodiments in any manner. Those skilled in the art can modify the embodiments of the present invention in various ways without departing from the meaning of the present invention, and such modifications are also be included within the scope of the present invention. Unless otherwise specified herein, all technical terms and scientific terms used in the present specification have the same meaning as those generally understood by those skilled in the art to which the present invention pertains.

### (Enteropeptidase Inhibitor)

Enteropeptidase is a transmembrane serine protease specifically expressed on the luminal side of the duodenum, and has the physiological action of cleaving and activating trypsinogen in pancreatic juice into trypsin.

In the above-mentioned embodiments, “enteropeptidase inhibitor” refers to all compounds that have the action of inhibiting the function of the enteropeptidase. Many “enteropeptidase inhibitors” are already known in the art, and “enteropeptidase inhibitor” is recognized and widely used by those skilled in the art as a comprehensive technical term referring to this group of compounds.

In the above-mentioned embodiments, those skilled in the art can appropriately select a preferred enteropeptidase inhibitor from the viewpoint of effectiveness, safety and the like. Such enteropeptidase inhibitory action can be confirmed according to methods known in the art.

Examples of such enteropeptidase inhibitor include the compounds described in WO 2015/122187, WO 2016/158788, WO 2016/104630, WO 2011/071048, WO 2019/088270, and WO 2019/216742, salts thereof, and hydrates thereof.

As a preferred enteropeptidase inhibitor here, one selected from a compound described in WO 2015/122187 or a salt thereof, or a hydrate thereof can be mentioned, a more preferred enteropeptidase inhibitor is a compound or a salt thereof, or a hydrate thereof selected from a compound represented by the formula (I):

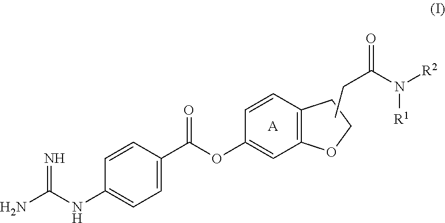

wherein
ring A is a benzene ring optionally substituted by 1 to 3 substituents selected from a halogen atom and a C₁₋₆ alkyl group;
R¹ is a hydrogen atom, or a C₁₋₆ alkyl group substituted by COOH, and
R² is a C₁₋₆ alkyl group substituted by 1 or 2 COOH, and further optionally substituted by SO₃H,
   described in WO 2015/122187, or a salt thereof, or a hydrate thereof,
a further preferred enteropeptidase inhibitor is a compound or a salt thereof, or a hydrate thereof selected from
N-(((3S)-6-((4-carbamimidamidobenzoyl)oxy)-2,3-dihydro-1-benzofuran-3-yl)acetyl)-L-aspartic acid (Example 1),
N-(((3R)-6-((4-carbamimidamidobenzoyl)oxy)-2,3-dihydro-1-benzofuran-3-yl)acetyl)-L-aspartic acid (Example 2),
N-((6-((4-carbamimidamidobenzoyl)oxy)-4-methyl-2,3-dihydro-1-benzofuran-3-yl)acetyl)-L-aspartic acid (Example 3),
   described in WO 2015/122187, or a salt thereof, or a hydrate thereof,
   a furthermore preferred enteropeptidase inhibitor is N-(((3S)-6-((4-carbamimidamidobenzoyl)oxy)-2,3-dihydro-1-benzofuran-3-yl)acetyl)-L-aspartic acid or a salt thereof, or a hydrate thereof, and
   the most preferred enteropeptidase inhibitor is N-(((3S)-6-((4-carbamimidamidobenzoyl)oxy)-2,3-dihydro-1-benzofuran-3-yl)acetyl)-L-aspartic acid hydrate (described in the above-mentioned WO 2015/122187, Example 12).

In the present specification, examples of the “halogen atom” include fluorine, chlorine, bromine, and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, and 2-ethylbutyl.

In the above-mentioned embodiment, the “enteropeptidase inhibitor” can also be used in either free form or in the form of a salt thereof (preferably, a pharmaceutically acceptable salt thereof). Those skilled in the art can carry out the present invention by selecting an appropriate form according to the nature of the “enteropeptidase inhibitor” to be used.

Pharmaceutically acceptable salts include, for example, salts with inorganic acids such as hydrochloride, hydrobromide, sulfate, and phosphate, salts with organic acids such as acetate, fumarate, oxalate, citrate, methanesulfonate, benzenesulfonate, tosylate, and maleate, salts with bases such as alkali metal salts such as sodium salt and potassium salt, and alkaline earth metal salts such as calcium salt, and salts with amino acids such as glycine salt, lysine salt, arginine salt, ornithine salt, glutamate, and aspartate.

When an enteropeptidase inhibitor has isomers such as optical isomer, stereoisomer, positional isomer, and rotational isomer, it can be used as one of the isomers or as a mixture thereof. When optical isomers exist in the enteropeptidase inhibitor, it is preferable to use the optical isomers separated from the racemate. These isomers can be obtained as individual optical isomers by synthesis methods known per se, separation methods (e.g., concentration, solvent extraction, column chromatography, recrystallization, etc.), and optical resolution methods (e.g., fractional recrystallization, chiral column chromatography, diastereomerization, etc.).

Enteropeptidase inhibitors may be crystalline, and may be a single crystalline form or a mixture of crystalline forms. Crystals can be produced by crystallization using crystallization methods known per se.

Enteropeptidase inhibitors may be solvates (e.g., hydrates, etc.) or non-solvates (e.g., nonhydrates, etc.), and both can be used in the embodiments described above.

Enteropeptidase inhibitors can also be used in the form of prodrugs thereof. Those skilled in the art can design such prodrugs as appropriate, and such prodrugs can be produced by methods known per se. The prodrug may be converted into the desired enteropeptidase inhibitor under physiological conditions, as described in Hirokawa Shoten, 1990, “Drug Development”, Vol. 7, Molecular Design, pp. 163-198.

### (Function of Enteropeptidase Inhibitor)

As described in detail in the Examples section below, it has become clear that enteropeptidase inhibitors can suppress and/or improve cyst formation in the kidney (or kidney and liver) in subjects suffering from polycystic kidney disease.

Polycystic kidney disease is a disease mainly induced by the formation of numerous cysts in the kidney. Therefore, enteropeptidase inhibitors are effective in suppressing and/or improving the above-mentioned cyst formation in subjects suffering from polycystic kidney disease, that is, effective in suppressing cyst formation and/or reducing the number of cysts and/or reducing the size of cysts.

Here, “suppression of cyst formation” refers to the function of suppressing the progression of cyst formation and suppressing cyst formation itself, “reducing the number of cysts” refers to the function of eliminating some of the formed cysts, and “reducing the size of cysts” includes both the function of reducing the size of formed cysts and the function of suppressing the increase in the size of formed cysts.

Enteropeptidase inhibitors can suppress and/or improve kidney volume increase based on the above-mentioned suppression and/or improvement of cyst formation. Here, “suppression of kidney volume increase” refers to the function of stopping or delaying the progress of kidney volume increase, and “improvement of kidney volume increase” refers to the function of reducing the increased kidney volume.

Enteropeptidase inhibitors are useful, for example, in improving kidney function by affording the above-mentioned various functions.

### (Target Disease of Enteropeptidase Inhibitor)

Because enteropeptidase inhibitors afford the above-mentioned functions, they can be used for the prevention and/or treatment of polycystic kidney disease. Furthermore, they can also be used for the prevention and/or treatment of polycystic liver disease which is a complication of polycystic kidney disease.

Here, examples of polycystic kidney disease include ADPKD, ARPKD, and the like.

“Prevention” includes preventing the onset (or recurrence) of the disease (the entire pathology or symptoms, or one or more pathologies or symptoms) and delaying the onset (or recurrence) of the disease. An “effective amount for prevention” refers to a dose of enteropeptidase inhibitor sufficient to achieve such goal.

“Treatment” includes curing the disease (the entire pathology or symptoms, or one or more pathologies or symptoms), improving the disease, and suppressing the progress of the severity of the disease. An “effective amount for treatment” refers to a dose of enteropeptidase inhibitor sufficient to achieve such goal.

### (Subject of Administration)

In the above-mentioned embodiments, a preferred enteropeptidase inhibitor is appropriately selected and used from the viewpoint of efficacy, safety, and the like. Therefore, enteropeptidase inhibitors can be safely administered to mammals (e.g., human, monkey, dog, horse, etc.) (especially to humans).

### (Administration Route)

Enteropeptidase inhibitors can be administered orally or parenterally (e.g., administered intravenously, by drip infusion, intramuscularly, subcutaneously, intraorganically, intranasally, intradermally, transdermally, by eye drops, intracerebrally, intrarectally, intravaginally, intraperitoneally, and to the lesion) alone or as a pharmaceutical composition.

### (Dosage)

The dosage of enteropeptidase inhibitors varies depending on the subject of administration, route of administration, and the age and symptoms of the subject of administration, but is not particularly limited. For example, the dosage, when administered orally, is 1 to 750 mg, preferably 10 to 500 mg, of the enteropeptidase inhibitor as the active ingredient. This dosage can be administered 1 to 3 times a day. When administered in the form of a sustained-release preparation, it is also possible to administer the preparation every other day or at longer intervals, corresponding to the dosage.

### (Administration Form)

Enteropeptidase inhibitors can be used in either alone or in the form of a pharmaceutical composition containing the enteropeptidase inhibitor as an active ingredient (in one embodiment, as a single active ingredient) together with a pharmaceutically acceptable carrier.

In the present specification, both forms are also collectively referred to as “medicament”.

Examples of such pharmaceutical composition include tablets (including sugar-coated tablet, film-coated tablet, sublingual tablet, orally disintegrating tablet, buccal tablet, etc.), pills, powders, granules, capsules (including soft capsule and microcapsule), lozenges, syrups, liquids, emulsions, suspensions, controlled-release preparations (e.g., immediate-release preparation, sustained-release preparation, sustained-release microcapsule), aerosols, films (e.g., orally disintegrating film, oral mucosal adhesive film), injections (e.g., subcutaneous injection, intravenous injections (e.g., bolus injection), intramuscular injection, intraperitoneal injection), drip infusions, transdermal preparations, ointments, lotions, patches, suppositories (e.g., anal suppository, vaginal suppository), pellets, nasal formulations, pulmonary formulations (inhalants), eye drops, and the like.

As “pharmaceutically acceptable carriers”, various carriers conventionally used in the field of formulation technology can be used. Specific examples of “pharmaceutically acceptable carriers” include, in solid preparations, excipients (e.g., lactose, sucrose, D-mannitol, starch, corn starch, crystalline cellulose, light anhydrous silicic acid, etc.), lubricants (e.g., magnesium stearate, talc, colloidal silica, etc.), binders (e.g., crystalline cellulose, sucrose, D-mannitol, dextrin, hydroxypropylcellulose, hydroxypropylmethylcellulose, polyvinylpyrrolidone, starch, sucrose, gelatin, methylcellulose, sodium carboxymethylcellulose, etc.) and disintegrants (e.g., starch, carboxymethylcellulose, calcium carboxymethylcellulose, sodium carboxymethyl starch, L-hydroxypropylcellulose, etc.).

In liquid preparations, solvents (e.g., water for injection, isotonic brine, alcohol, propylene glycol, macrogol, sesame oil, etc.), solubilizers (e.g., polyethylene glycol, propylene glycol, D-mannitol, benzyl benzoate, ethanol, triethanolamine, sodium carbonate, sodium citrate, etc.), suspending agents (e.g., surfactants such as stearyltriethanolamine, sodium lauryl sulfate, laurylaminopropionic acid, lecithin, benzalkonium chloride, glyceryl monostearate; hydrophilic polymers such as polyvinyl alcohol, polyvinylpyrrolidone, sodium carboxymethylcellulose, methylcellulose, hydroxymethylcellulose, hydroxypropylcellulose, etc.), isotonic agents (e.g., glucose, D-sorbitol, sodium chloride, glycerin, D-mannitol, etc.), buffers (e.g., buffers such as phosphates and citrates), and analgesics (e.g., benzyl alcohol, etc.) can be used.

When necessary, formulation additives such as preservatives (e.g., para-hydroxybenzoic acid esters, chlorobutanol, benzyl alcohol, sorbic acid, etc.), antioxidants (e.g., sulfites, ascorbic acid, α-tocopherol, etc.), colorants, sweeteners, and the like may be further added.

While pharmaceutical compositions may vary depending on the dosage form, administration method, carrier, and the like, but they can be manufactured by adding an enteropeptidase inhibitor at a rate of generally 0.01 to 99% (w/w), preferably 0.1 to 85% (w/w), relative to the total amount of the preparation. The pharmaceutical composition can be manufactured by methods conventionally used in the field of formulation technology, depending on the form thereof. The pharmaceutical composition may be molded into a release-controlled preparation such as a rapid-release preparation or a sustained-release preparation containing the active ingredient.

(Combined Use with Other Agents)

In practicing the above-mentioned embodiment, enteropeptidase inhibitors can be used in combination with other drugs for the prevention and/or treatment of the target disease, and excellent preventive and/or therapeutic effects can be expected by combined use with other drugs. Furthermore, it can be expected that the dose of other drugs can be reduced through such combination therapy, thereby reducing the side effects they have.

The drugs that can be used in combination with enteropeptidase inhibitors (hereinafter sometimes abbreviated as “combination drugs”) can be appropriately selected in consideration of the type of the disease of the patient, the severity of the symptoms, and the like. For example, combined use with vasopressin V2 receptor antagonists (e.g., tolvaptan), somatostatin analogs (e.g., octreotide, pasireotide, etc.), retinoic acid receptor α subtype agonists (e.g., tamibarotene), microRNA inhibitors (e.g., falabrusen), anti-pregnancy-related plasma protein-A monoclonal antibodies (e.g., ABBV-CLS-628), angiotensin-converting enzyme inhibitors (e.g., captopril, enalapril, delapril, etc.), angiotensin II antagonists (e.g., candesartan, losartan, eprosartan, valsartan, telmisartan, irbesartan, olmesartan, azilsartan, etc.), SGLT2 inhibitors (e.g., dapagliflozin, empagliflozin, ipragliflozin, canagliflozin, etc.), and biguanide antidiabetic drugs (e.g., metformin, etc.) is possible.

The administration form of the concomitant drug is not particularly limited, and the enteropeptidase inhibitor and the concomitant drug may be combined at the time of administration. For example, the following forms can be adopted: (1) administration of a preparation containing the enteropeptidase inhibitor and the concomitant drug in combination; (2) simultaneous or separate administration of two preparations obtained by separately formulating the enteropeptidase inhibitor and the concomitant drug via the same route of administration; or (3) simultaneous or separate administration of two preparations obtained by separately formulating the enteropeptidase inhibitor and the concomitant drug via different routes of administration. A preferred form can be appropriately selected according to the actual situation in the healthcare setting.

The preparation containing the above-mentioned enteropeptidase inhibitor and concomitant drug in combination can be appropriately manufactured by a person skilled in the art in accordance with the pharmaceutical composition containing the enteropeptidase inhibitor described above.

The dosage of the concomitant drug can be appropriately selected based on the doses used clinically. Furthermore, the mixing ratio of the enteropeptidase inhibitor and the concomitant drug can be appropriately selected depending on the disease or symptoms of the subject of administration, the route of administration, the type of concomitant drug to be used, and the like. Generally, the dosage can be appropriately determined based on the general clinical dose of the concomitant drug to be used, according to the actual situation in the healthcare setting.

Another embodiment of the present invention includes the following forms.
[C] A method for suppressing and/or improving cyst formation, or suppressing and/or improving kidney volume increase based on the suppression and/or improvement of cyst formation, in a subject suffering from polycystic kidney disease, comprising administering an effective amount of an enteropeptidase inhibitor to the subject.
[C-1] The method of the above-mentioned [C], for the improvement of kidney function.
[D] A method for suppressing and/or improving polycystic kidney disease, comprising administering an effective amount of an enteropeptidase inhibitor to a subject requiring the administration.
[D-1] The method of the above-mentioned [D], for preventing and/or treating polycystic kidney disease and polycystic liver disease as a complication thereof.
[D-2] The method of the above-mentioned [D] or [D-1], wherein the polycystic kidney disease is ADPKD or ARPKD.

For each technical matter that specifies the above-mentioned embodiments, for example, “enteropeptidase inhibitor”, “function afforded by the enteropeptidase inhibitor”, “target disease”, “subject of administration”, “dosage”, “administration route”, “administration form”, and the like, the descriptions of the corresponding technical matters in the aforementioned Embodiments A and B can be referred to.

### EXAMPLE

The present invention is explained in more detail in the following by referring to Examples, which do not limit the scope of the present invention in any manner. The reagents, apparatuses and materials used in the present invention are commercially available or can be appropriately prepared by those of ordinary skill in the art, unless particularly indicated.

### Example 1: Human Enteropeptidase Inhibitory Activity

In this example, the compound described in WO 2015/122187 was used. Compounds 1 to 11 of the examples described in the publication thereof (the compound of Example 12 is a hydrate of the compound of Example 1) have been shown to have superior enteropeptidase inhibitory action (Experimental Example 1).

### Example 2 Evaluation of Suppressive Action on Amino Acid Concentration Increase in Postprandial Blood Using ADPKD Model Mice

Pkd1^{flox/flox}:Mx1-Cre mice aged 10-12 days were administered 2.5 μg/g polyinosinic acid: polycytidylic acid (InvivoGen) intraperitoneally for 6 consecutive days to induce deletion of the Pkd1 gene. At any point between 28 and 90 days of age, the mice were fasted for 3 hr, and either a solution of test compound 12 (N-(((3S)-6-((4-carbamimidamidobenzoyl)oxy)-2,3-dihydro-1-benzofuran-3-yl)acetyl)-L-aspartic acid hydrate) as a representative enteropeptidase inhibitor described in the above-mentioned WO 2015/122187, suspended in 0.5% methylcellulose (compound administration group, 10 or 30 mg/kg), or a 0.5% methylcellulose solution (control group) was orally administered. Here, test compound 12 was a hydrate of test compound 1, and prepared so that the equivalent amount as test compound 1 was the target dose. After 1 hr, a 2.5 g/kg protein solution (SAVAS Whey Protein 100, Meiji Co., Ltd.) was orally administered. Blood samples were collected before administration of the protein solution and at 0.5 and 1.5 hr after the administration. Serum BCAA concentration was measured using an enzymatic method (BCAA assay kit, Cell Biolabs) as an indicator of amino acid absorption into the body. The area of the serum BCAA concentration-time curve (AUC) from before administration to 1.5 hr after administration was calculated, and the postprandial increase in blood amino acid concentration (%) was calculated using the following formula:

$$postprandial increase in blood amino acid concentration ( \% ) = ( AUC of target group \div AUC of control group ) \times 100$$

The results are shown in Table 1. The significance of each test group relative to the control group was evaluated by Dunnett's test, and the significance level relative to the control group is indicated as *:p<0.05, **:p<0.01.

TABLE 1

| test compound | dose | postprandial increase in blood |
|---|---|---|
| (Example No.) | (mg/kg) | amino acid concentration (%) |
| control | 0 | 100.0 ± 25.3 |
| 12 | 10 | 56.7 ± 25.3* |
| 12 | 30 | 43.6 ± 24.8** |

As is clear from Table 1, test compound 12 was shown to suppress the absorption of protein into the body after meals and to decrease blood amino acid concentration in ADPKD model mice.

### Example 3: Evaluation of Cyst Formation Suppressive Effect Using ADPKD Model Mice

Pkd1^{flox/flox}:Mx1-Cre mice aged 10 days were intraperitoneally administered 10 μg/g polyinosinic acid: polycytidylic acid (Sigma-Aldrich) for 6 consecutive days to induce deletion of the Pkd1 gene. Subsequently, from 4 weeks of age, they were fed with either a normal diet (MF, Oriental Yeast Co., Ltd.) or a diet mixed with test compound 12 as a representative enteropeptidase inhibitor described in the above-mentioned WO 2015/122187 at a rate of 0.003% and 0.01% (w/w) of the normal diet. Here, test compound 12 was a hydrate of test compound 1, and prepared so that the equivalent amount as test compound 1 was the target dose. At the end of the 4-week administration period, the mice were anesthetized, perfused with 4% paraformaldehyde, and the kidneys and livers were collected and weighed. The tissue weight/body weight (%) was calculated from the following formula using the body weight at necropsy:

$$tissue weight / body weight ( \% ) = ( tissue weight \div body weight ) \times 100$$

Furthermore, each tissue was embedded in paraffin, stained with hematoxylin and eosin, and stained section images were obtained using an all-in-one fluorescence microscope (BZ-X710, KEYENCE CORPORATION). The cystic index (CI) was calculated from the following formula:

$$CI ( \% ) = ( total cyst area \div total tissue area ) \times 100$$

Furthermore, kidney tissue was solubilized with homogenization buffer (20 mM Tris-HCl, 150 mM NaCl, 1 (v/v) % Triton X-100, protein inhibitor cocktail (Roche Life Sciences) and phosphatase inhibitor cocktail (Sigma-Aldrich)), and 100 μg of tissue fluid was separated by SDS-PAGE and transferred to a polyvinylidene fluoride (PVDF) membrane. The PVDF membrane was immersed in 5 (w/v) % skim milk for 1 hr, and then incubated overnight with a primary antibody against p-S6 (Ser235/236) or S6 (Cell Signaling Technology). Then, the PVDF membrane was incubated with a secondary antibody conjugated with horseradish peroxidase. After washing, the chemiluminescence obtained from the target protein using SuperSignal West Femto Maximum Sensitivity Substrate (Thermo Fischer Scientific) was detected using ImageQuant LAS4000 (GE Healthcare). The expression level of phosphorylated S6 protein was calculated from the following formula using the optical density of the bands obtained with the primary antibodies for p-S6 (Ser235/236) and S6:

$$p - S 6 / S 6 ( ratio ) = optical density of p - S 6 ( Ser 235 / 236 ) band / optical density of S 6 band$$

The results of tissue weight/body weight, CI, and p-S6/S6 (ratio) are shown in Table 2. The significance of each test group relative to the control group was evaluated by Dunnett's test, and the significance level relative to the control group is indicated as *:p<0.05, **:p<0.01.

TABLE 2

| | | test | test |
|---|---|---|---|
| test group | control | compound 12 | compound 12 |
| dose (%) | 0 | 0.003 | 0.01 |
| kidney weight/body | 3.3 ± 1.1 | 3.2 ± 1.2 | 2.1 ± 0.4** |
| weight (%) | | | |
| kidney CI (%) | 57.0 ± 11.7 | 52.3 ± 7.7 | 44.7 ± 7.6** |
| p-S6/S6 (ratio) | 0.95 ± 0.74 | 0.91 ± 0.81 | 0.20 ± 0.19* |
| liver weight/body | 5.9 ± 1.0 | 5.7 ± 0.9 | 4.4 ± 0.5* |
| weight (%) | | | |
| liver CI (%) | 17.6 ± 5.8 | 16.7 ± 5.4 | 12.8 ± 3.1** |

As is clear from Table 2, test compound 12 reduced p-S6/S6 which is an indicator of mTOR activation in ADPKD model mice, and was shown to suppress the progression of renal hypertrophy and renal cysts. Furthermore, in addition to the effects on the kidney, test compound 12 also showed a suppressive effect on the progression of hepatomegaly and hepatic cysts. These test results clearly demonstrate that test compound 12, and enteropeptidase inhibitors represented thereby, are useful for the prevention or treatment of ADPKD.

Furthermore, the above-mentioned test results suggest that enteropeptidase inhibitors are useful for the prevention and/or treatment of polycystic kidney disease, which is a disease mainly caused by cyst formation in the kidney, or polycystic liver disease as a complication thereof.

### INDUSTRIAL APPLICABILITY

The present invention provides, as embodiments thereof, a medicament containing an enteropeptidase inhibitor as an active ingredient, for suppressing and/or improving cyst formation, or suppressing and/or improving kidney volume increase based on the suppression and/or improvement of cyst formation, in a subject suffering from polycystic kidney disease; a medicament containing an enteropeptidase inhibitor as an active ingredient, for preventing and/or treating polycystic kidney disease, or polycystic liver disease as a complication thereof; a method for suppressing and/or improving cyst formation, or suppressing and/or improving kidney volume increase based on the suppression and/or improvement of cyst formation, in a subject suffering from polycystic kidney disease, containing administering an effective amount of an enteropeptidase inhibitor to the subject; a method for preventing and/or treating polycystic kidney disease, containing administering an effective amount of an enteropeptidase inhibitor to a subject requiring the administration; and the like, and is useful in the pharmaceutical field.

1. A method for suppressing and/or improving cyst formation, or suppressing and/or improving kidney volume increase based on the suppression and/or improvement of cyst formation, in a subject suffering from polycystic kidney disease, comprising administering an effective amount of an enteropeptidase inhibitor to the subject.

2. The method according to claim 1, for the improvement of kidney function.

3. A method for preventing and/or treating polycystic kidney disease, comprising administering an effective amount of an enteropeptidase inhibitor to a subject requiring the administration.

4. The method according to claim 3, for preventing and/or treating polycystic kidney disease and polycystic liver disease as a complication thereof.

5. The method according to claim 4, wherein the polycystic kidney disease is ADPKD (autosomal dominant polycystic kidney disease) or ARPKD (autosomal recessive polycystic kidney disease).

6. The method according to claim 1, wherein the enteropeptidase inhibitor is a compound described in WO 2015/122187, WO 2016/158788, WO 2016/104630, WO 2011/071048, WO 2019/088270, or WO 2019/216742, or a salt thereof, or a hydrate thereof.

7. The method according to claim 1, wherein the enteropeptidase inhibitor is a compound described in WO 2015/122187, or a salt thereof, or a hydrate thereof.

8. The method according to claim 1, wherein the enteropeptidase inhibitor is a compound represented by the formula (I): 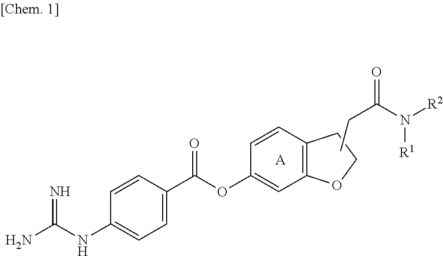
wherein
ring A is a benzene ring optionally substituted by 1 to 3 substituents selected from a halogen atom and a C₁₋₆ alkyl group;
R¹ is a hydrogen atom, or a C₁₋₆ alkyl group substituted by COOH, and
R² is a C₁₋₆ alkyl group substituted by 1 or 2 COOH, and further optionally substituted by SO₃H,
described in WO 2015/122187, or a salt thereof, or a hydrate thereof.

9. The method according to claim 1, wherein the enteropeptidase inhibitor is a compound selected from the group consisting of
N-(((3S)-6-((4-carbamimidamidobenzoyl)oxy)-2,3-dihydro-1-benzofuran-3-yl)acetyl)-L-aspartic acid,
N-(((3R)-6-((4-carbamimidamidobenzoyl)oxy)-2,3-dihydro-1-benzofuran-3-yl)acetyl)-L-aspartic acid, and
N-((6-((4-carbamimidamidobenzoyl)oxy)-4-methyl-2,3-dihydro-1-benzofuran-3-yl)acetyl)-L-aspartic acid,
described in WO 2015/122187, or a salt thereof, or a hydrate thereof.

10. The method according to claim 1, wherein the enteropeptidase inhibitor is N-(((3S)-6-((4-carbamimidamidobenzoyl)oxy)-2,3-dihydro-1-benzofuran-3-yl)acetyl)-L-aspartic acid or a salt thereof, or a hydrate thereof.

11. The method according to claim 1, wherein the enteropeptidase inhibitor is N-(((3S)-6-((4-carbamimidamidobenzoyl)oxy)-2,3-dihydro-1-benzofuran-3-yl)acetyl)-L-aspartic acid hydrate.

12. The method according to claim 3, wherein the enteropeptidase inhibitor is a compound described in WO 2015/122187, WO 2016/158788, WO 2016/104630, WO 2011/071048, WO 2019/088270, or WO 2019/216742, or a salt thereof, or a hydrate thereof.

13. The method according to claim 3, wherein the enteropeptidase inhibitor is a compound described in WO 2015/122187, or a salt thereof, or a hydrate thereof.

14. The method according to claim 3, wherein the enteropeptidase inhibitor is a compound represented by the formula (I): 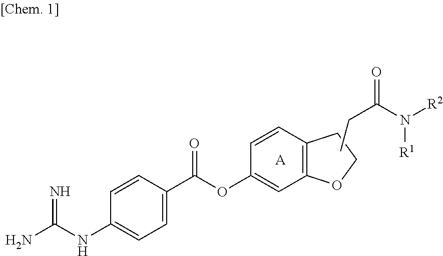
wherein
ring A is a benzene ring optionally substituted by 1 to 3 substituents selected from a halogen atom and a C₁₋₆ alkyl group;
R¹ is a hydrogen atom, or a C₁₋₆ alkyl group substituted by COOH, and
R² is a C₁₋₆ alkyl group substituted by 1 or 2 COOH, and further optionally substituted by SO₃H,
described in WO 2015/122187, or a salt thereof, or a hydrate thereof.

15. The method according to claim 3, wherein the enteropeptidase inhibitor is a compound selected from the group consisting of
N-(((3S)-6-((4-carbamimidamidobenzoyl)oxy)-2,3-dihydro-1-benzofuran-3-yl)acetyl)-L-aspartic acid,
N-(((3R)-6-((4-carbamimidamidobenzoyl)oxy)-2,3-dihydro-1-benzofuran-3-yl)acetyl)-L-aspartic acid, and
N-((6-((4-carbamimidamidobenzoyl)oxy)-4-methyl-2,3-dihydro-1-benzofuran-3-yl)acetyl)-L-aspartic acid,
described in WO 2015/122187, or a salt thereof, or a hydrate thereof.

16. The method according to claim 3, wherein the enteropeptidase inhibitor is N-(((3S)-6-((4-carbamimidamidobenzoyl)oxy)-2,3-dihydro-1-benzofuran-3-yl)acetyl)-L-aspartic acid or a salt thereof, or a hydrate thereof.

17. The method according to claim 3, wherein the enteropeptidase inhibitor is N-(((3S)-6-((4-carbamimidamidobenzoyl)oxy)-2,3-dihydro-1-benzofuran-3-yl)acetyl)-L-aspartic acid hydrate.
